(19) United States
(12) Plant Patent Application Publication
(10) Pub. No.: US 2003/0033649 P1
(43) Pub. Date: Feb. 13, 2003

(54) NIEREMBERGIA PLANT NAMED 'SUNNICOBU'

(76) Inventor: YASUYUKI MURAKAMI, GAMOU-GUN (JP)

Correspondence Address: BURNS DOANE SWECKER AND MATHIS, GEORGE MASON BUILDING, WASHINGTON AND PRINCE STREETS, P O BOX 1404, ALEXANDRIA, VA, 223131404

(21) Appl. No.: 09/334,501

(22) Filed: Jun. 17, 1999

(51) Int. Cl. A01H 5/00

(52) U.S. Cl. PLT/263

(57) ABSTRACT

Disclosed herein is new Nierembergia plant named ‘SUNNICOBU’, having a compact and tufted habit with long internode stem. The stem color varies from grayish olive to grayish brown. Flowers are single and small. The petal is light purple with moderate purple center portion. The bottom color of the corolla throat is vivid yellow and the outside color of corolla tube is pale yellow green. The plant has a high resistance to rain, heat, diseases especially powdery mildew.

### BACKGROUND OF THE VARIETY

The present invention relates to a new and distinct variety of Nierembergia PLANT NAMED ‘SUNNICOBU’. ‘SUNNICOBU’ is the first vegetative propagated variety which was developed by specific hybridization, between Nierembergia gracilis and Nierembergia caerulea. This variety is a breakthrough in breeding, as it combines flowers of light purple with darker center, compact and tufted habit, good winter hardiness, and high resistance to heat, rain, diseases especially powdery mildew.

Nierembergia genus is Solanaceae plants and herbaceous perennials or semi-woody shrub. Hardy or half-hardy plants. There are 30 species, in Mexico to South America, around Chile and Argentina. Stems hanging down or lying flat or erect. Simple leaves and phyllotaxy is alternate or dispersed. Flowers arise from axillary. Inflorescence type is flower solitary or cyme. Flower color is white or light purple, or unusually purple. Occasionally center color is purple. Bottom color of corolla throat is yellow. Calyx is tubular or funnel-shaped. Flower shape is funnel-form or campanulate or salver-form. Tube is thin. Throat spreads widely. Rim separates into 5. Number of stamen is 5 attached at the inside of tube. 4 of 5 stamens make pairs and one is a small staminode. Capsule has 2 carpels and splits open. Basic number of chromosome is x=9. Long day, summer flowering plant.

The female parent used in the crossing of ‘SUNNICOBU’ was Nierembergia gracilis, which is a wild species of Nierembergia plant native to Argentina and introduced to Japan in 1992. It is a compact and tufted bush. The flowers are single, medium to small size and white petals. The leaf is lanceolate. Nierembergia gracilis is vulnerable to the rain, heat, and powdery mildew.

The pollen parent use in the crossing of ‘SUNNICOBU’ was Nierembergia caerulea, which is a wild species of Nierembergia genus and cultivated in England and introduced to Japan in 1992. It has erect habit plant, less branching. The flowers are single, medium size and have vivid purple petals. The leaf is lanceolate and waving. Nierembergia caerulea has good winter hardiness and a resistance to rain, diseases and pests.

First of all, two wild types of Nierembergia genus, Nierembergia gracilis and Nierembergia caerulea, were introduced to Japan in 1992. The seedlings of both wild types Nierembergia genus were grown at the Hakushu Nursery Center of SUNTORY Ltd., since spring of 1994, and few seedlings were selected from both lines. Under green-house condition, the controlled crossing with seedlings of Nierembergia gracilis (♀) and that of Nierembergia caerulea (♀) was conducted in June 1995. From seedlings which were obtained through this crossing, 10 seedlings were selected in view of compact and tufted plant habit and flower color in spring of 1996 in greenhouse condition. The botanical characteristics of the finally selected 10 varieties were then examined. They were tested at the Oumi Nursery Center of SUNTORY Ltd. from spring to autumn in 1996. As a result, one variety was selected which met our criteria and was uniform and stable in its characteristics. This new variety of Nierembergia genus plant was named ‘SUNNICOBU’ (Nierembergia hybrida).

These wild type of Nierembergia genes plants, Nierembergia gracilis and Nierembergia caerulea, and ‘SUNNICOBU’ are presently maintained at the Oumi Nursery Center of SUNTORY Ltd., residing at 863-1, Aza-Iketani, Oomori-cho, Youkaichi-shi, Shiga-ken, Japan.

In the following description, the color-coding is in accordance with the Horticultural Color Chart of The Royal Horticultural Society, London, England (R.H.S. Color Chart), and the Inter-Society color Council-Nation Bureau of Standard Color Name (I.S.C.C.-N.B.S. Color Name). A color chart based on The Japan Color Standard for Horticultural Plant (J.H.S. Color Chart) is also added for reference.

The botanical characteristics of the female parent plant Nierembergia gracilis used in the crossing of ‘SUNNICOBU’ as follows.

Plant:

Growth habit.—Tufted.

Height.—12 cm.

Spread.—26 cm.

Stem:

Thickness.—0.9 mm.

Color.—Moderate olive green (R.H.S. 147A, J.H.S. 3508).

Branching.—Abundant.

Pubescence.—Present.

Length of internode.—8 mm.

Leaf:

Whole shape.—Lanceolate.

Apex shape.—Acuminate.

Base shape.—Cuneate.

Length.—25 mm.

Width.—1.9 mm.

Thickness.—0.3 mm.

Color of upper side.—Moderate yellow green (R.H.S. 137B, J.H.S. 3712).

Color of reverse side.—Moderate yellow green (R.H.S. 138A, J.H.S. 3513).

Pubescence.—Sparse.

Attachment angle to stem.—Horizontal.

Flower:

Facing direction.—Upward.

Type.—Single.

Shape.—Rotate, five-fissured limb

Shape of petal tip.—Obtuse.

Waving of petal.—Weak.

Lobation of petal.—Shallow.

Diameter of petal.—30 mm.

Diameter of tube.—0.8 mm.

Length of tube.—14 mm.

Color of petal.—White (R.H.S. 155D, J.H.S. -01) with light purple center portion (R.H.S. 85C, J.H.S. 8303).

Bottom color of corolla throat.—Vivid yellow (R.H.S. 12A, J.H.S. 2506).

Outside color of corolla throat.—Pale yellow green (R.H.S. 4D, J.H.S. 3303).

Flowering habit.—Middle.

Pistil:

Number.—1.

Type.—Normal.

Color of stigma.—Strong yellow green (R.H.S. 144A, J.H.S. 3507).

Stamen:

Number.—5.

Type.—Syngenesious.

Color of anther.—Vivid yellow (R.H.S. 12A, J.H.S. 2506).

Hardiness:

Winter.—Good.

Rain.—Poor.

Heat.—Poor.

Resistance:

Disease.—Poor.

Insect.—Excellent.

And the botanical characteristics of the male parent plant Nierembergia caerulea used in the crossing of ‘SUNNICOBU’ as follows.

Plant:

Growth habit.—Upright.

Height.—15 cm.

Spread.—18 cm.

Stem:

Thickness.—0.7 mm.

Color.—Moderate olive green (R.H.S. 147A, J.H.S. 3508).

Branching.—Medium.

Pubescence.—Present.

Length of internode.—9 mm.

Leaf:

Whole shape.—Lanceolate.

Apex shape.—Acuminate.

Base shape.—Cuneate.

Length.—15 mm.

Width.—1.6 mm.

Thickness.—0.3 mm.

Color of upper side.—Moderate yellow green (R.H.S. 137A, J.H.S. 3712).

Color of reverse side.—Moderate yellow green (R.H.S. 137B, J.H.S. 3712).

Pubescence.—Sparse.

Attachment angle to stem.—Upright.

Flower:

Facing direction.—Upward.

Type.—Single.

Shape.—Rotate, five-fissured limb.

Shape of petal tip.—Obtuse.

Waving of petal.—Weak.

Lobation of petal.—Shallow.

Diameter of petal.—33 mm.

Diameter of tube.—0.8 mm.

Length of tube.—16 mm.

Color of petal.—Vivid purple (R.H.S. 88A, J.H.S. 8306).

Bottom Color of corolla throat.—Brilliant greenish. yellow (R.H.S. 9B, J.H.S. 2704).

Outside color of corolla throat.—Light gray (R.H.S. 202C, J.H.S. -2).

Flowering habit.—Late.

Pistil:

Number.—1.

Type.—Normal.

Color of stigma.—Strong yellow green (R.H.S. 144A, J.H.S. 3507).

Stamen:

Number.—5.

Type.—Syngenesious.

Color of anther.—Vivid yellow (R.H.S. 12A, J.H.S. 2506).

Hardiness:

Winter.—Excellent.

Rain.—Excellent.

Heat.—Good.

Resistance:

Disease.—Excellent.

Insect.—Excellent.

The botanical characteristics of the similar variety ‘MONTEBRANCO’ used for examination as a comparison are as follows.

Plant:

Growth habit.—Tufted.

Height.—9 cm.

Spread.—18 cm.

Stem:

Thickness.—0.8 mm.

Color.—Moderate olive green (R.H.S. 146A, J.H.S. 3508).

Branching.—Abundant.

Pubescence.—Present.

Length of internode.—8 mm.

Leaf:

Whole shape.—Linear.

Apex shape.—Acuminate.

Base shape.—Cuneate.

Length.—15 mm.

Width.—1.1 mm.

Thickness.—0.3 mm.

Color of upper side.—Moderate yellow green (R.H.S. 138A, J.H.S. 3513).

Color of reverse side.—Moderate yellow green (R.H.S. 137C, J.H.S. 3712).

Pubescence.—Sparse.

Attachment angle to stem.—Horizontal.

Flower:

Facing direction.—Upward.

Type.—Single.

Shape.—Rotate, five-fissured limb.

Shape of petal tip.—Obtuse.

Waving of petal.—Weak.

Lobation of petal.—Shallow.

Diameter of petal.—27 mm.

Diameter of tube.—0.8 mm.

Length of tube.—15 mm.

Color of petal.—White (R.H.S. 155D, J.H.S. -01).

Bottom color of corolla throat.—Vivid yellow (R.H.S. 12A, J.H.S. 2506).

Outside color of corolla throat.—Pale yellow green (R.H.S. 4D, J.H.S. 3303).

Flowering habit.—Middle.

Pistil:

Number.—1.

Type.—Normal.

Color of stigma.—Strong yellow green (R.H.S. 144A, J.H.S. 3507).

Stamen:

Number.—5.

Type.—Syngenesious.

Color of anther.—Vivid yellow (R.H.S. 12A, J.H.S. 2506).

Hardiness:

Winter.—Good.

Rain.—Poor.

Heat.—Poor.

Resistance:

Disease.—Poor.

Insect.—Excellent.

This new and distinct variety of Nierembergia plant, ‘SUNNICOBU’, was asexual reproduced by cutting at the aforementioned the Oumi Nursery Center of SUNTORY Ltd., and the homogeneity and stability thereof were confirmed.

### SUMMARY OF THE VARIETY

‘SUNNICOBU’ has stem that is 0.9 mm in diameter, medium size in its group, and has upright growth habit. The stem is flexible, therefore it extends laterally and new stems appear from the center. Due to that habit, plant makes medium size round shape, averaging 10 cm in height and 15 cm in width at the blooming time of the 5th to the 10th flower. The Stem color varies from grayish olive (R.H.S 152B, J.H.S 2917) to grayish brown (R.H.S 200C, J.H.S 1619) as against the stem of ‘MONTEBRANCO’ which shows no anthocyanin coloration and moderate olive green. The stem has sparse white pubescence, and the pubescence is slightly denser than that of ‘MONTEBRANCO’.

The number of the first lateral branches is about 6, the second lateral branches are about 3 at the blooming time of the 5th to the 10th flower, that is similar to that of ‘MONTEBRANCO’. Internode is 13 mm at the blooming time of the first flower. The internode is longer than that of ‘MONTEBRANCO’.

Leaves are lanceolate, 21 mm long, 2 mm width, and 0.4 mm thickness. The Leaf size is larger than that of ‘MONTEBRANCO’.

Flowers face upward. Corolla tube is very narrow, 16 mm length (from sepal to petal) and 0.8 mm in diameter. That is peculiar to Nierembergia genus plant. Flower is single and flower size is 33 mm in diameter. Petal color is light purple (J.H.S. 8303) with moderate purple (J.H.S. 8309) center portion. That is a distinctive characteristic of this new variety ‘SUNNICOBU’. Bottom color of corolla throat is vivid yellow (J.H.S 2506, R.H.S 9A, 9B, 12A, 13A). Flower-stem is 9 mm long and 0.5 mm wide, and longer and thicker than that of ‘MONTEBRANCO’. The structure of flower is the same as the varieties of this Nierembergia genus. Blooming period is from May extending to October.

It has good winter hardiness, can survive at −10° C. Also it has higher resistance to heat and rain than ‘MONTEBRANCO’. However, some damage to flower petals occurs under heavy rain due to its thinness, which is peculiar to this Nierembergia genus. High resistance to powdery mildew and almost on damage by diseases and pests.

### BRIEF DESCRIPTION OF THE DRAWING

FIG. 1 is a photograph giving a partial view of the new variety of Nierembergia plant ‘SUNNICOBU’ planted in the ground.

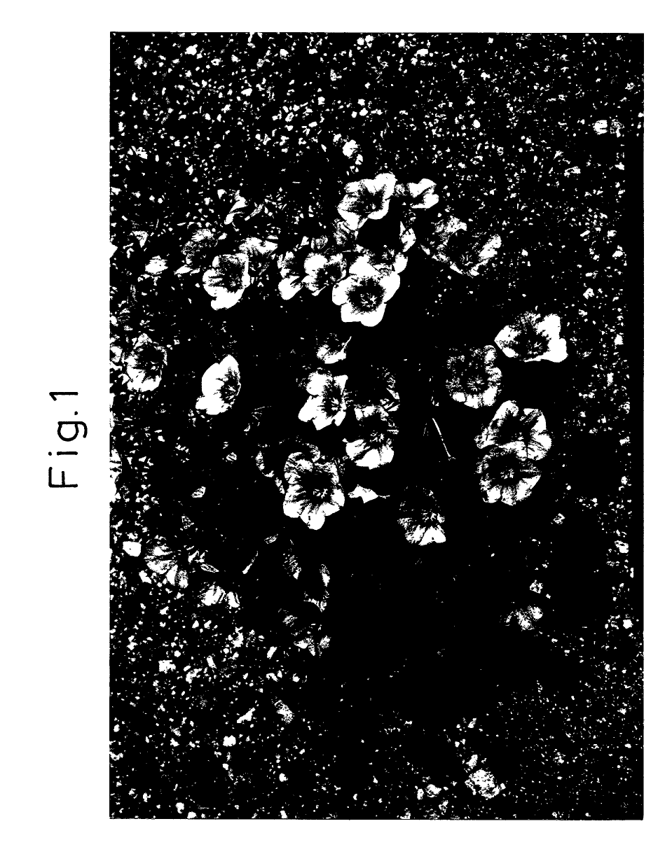

FIG. 2 is a photograph of flowers of the new variety of Nierembergia plant ‘SUNNICOBU’.

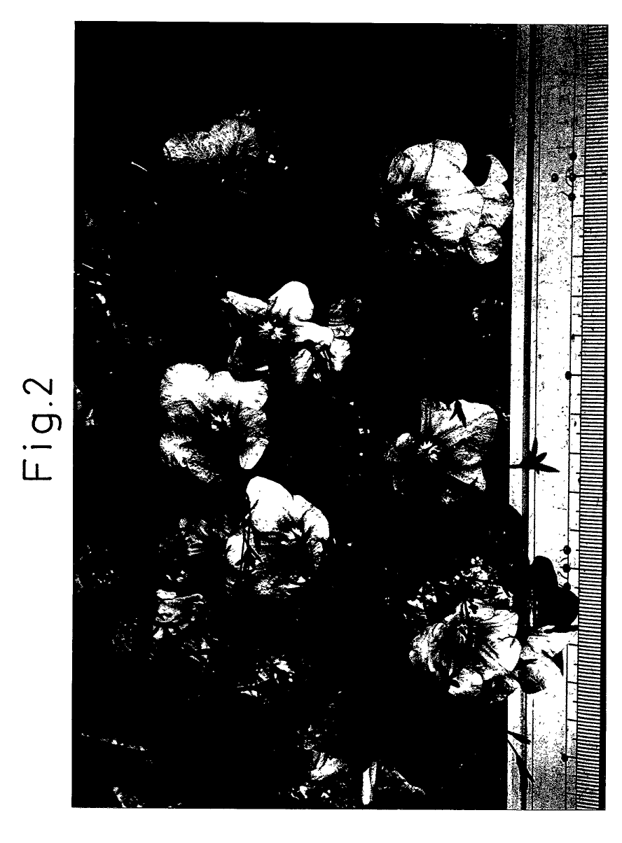

### DESCRIPTION OF THE VARIETY

The botanical characteristics of this new and distinct variety of Nierembergia plant named ‘SUNNICOBU’ are as follows.

Plant:

Growth habit.—Tufted.

Height.—10 cm.

Spread.—28 cm.

Stem:

Thickness.—0.9 mm.

Color.—Grayish olive—Grayish brown (R.H.S 152B 200C, J.H.S 2917- 1619).

Branching.—Abundant.

Pubescence.—Dense.

Length of internode.—13 mm.

Leaf:

Whole shape.—Lanceolate.

Apex shape.—acuminate.

Base shape.—Cuneate.

Length.—21 mm.

Width.—2.0 mm.

Thickness.—0.4 mm.

Color of upper side.—Moderate yellow green (R.H.S 137B, J.H.S 3712).

Color of reverse side.—Moderate yellow green (R.H.S 137C, J.H.S 3712).

Pubescence.—Sparse.

Attachment angle to stem.—Upright.

Flower:

Facing direction.—Upward.

Type.—Single.

Shape.—Rotate, five-fissured limb.

Shape of petal tip.—Obtuse.

Waving of petal.—Weak.

Lobation of petal.—Shallow.

Diameter of petal.—33 mm.

Diameter of tube.—0.8 mm.

Length of tube.—16 mm.

Color of petal.—Light purple (R.H.S 85B, J.H.S 8303) with moderate purple center portion (R.H.S 86D, J.H.S 8309).

Bottom color of corolla throat.—Vivid yellow (R.H.S 12A, J.H.S 2506).

Outside color of corolla throat.—Pale yellow green (R.H.S 157A, J.H.S 3303).

Flowering habit.—Middle.

Pistil:

Number.—1.

Type.—Normal.

Color of stigma.—Strong yellow green - (R.H.S 144A, J.H.S 3507).

Stamen:

Number.—5.

Type.—syngenesious.

Color of anther.—Vivid yellow (R.H.S 12A, J.H.S 2506).

Hardiness:

Winter.—Excellent.

Rain.—Excellent,

Heat.—Excellent.

Resistance:

Disease.—Excellent.

Insect.—Excellent.

This new variety of Nierembergia plant “SUNNICOBU” is most suitable for flower bedding and potting, and also excellent for ground cover.

1. A new and distinct variety of Nierembergia plant named ‘SUNNICOBU’, substantially as herein illustrated and described, characterized particularly as to novelty by (A) being a compact and tufted plant habit having long internode stem, (B) stem of which color varies from grayish olive to grayish brown (C) flowers which are single and small, the petals are light purple with moderate purple center portions, the bottom color of the corolla throat is vivid yellow and the outside color of the corolla tube is pale yellow green and (D) has a high resistance to rain, heat, and diseases, especially powdery mildew.
